(19) United States
(12) Patent Application Publication
(10) Pub. No.: US 2026/0157818 A1
(43) Pub. Date: Jun. 11, 2026

(54) SURGICAL TRAY

(71) Applicant: Thomas J. BURNS, Emmitsburg, MD (US)

(72) Inventor: Thomas J. BURNS, Emmitsburg, MD (US)

(73) Assignee: Keane Innovations, LLC, Emmitsburg, MD (US)

(21) Appl. No.: 19/414,392

(22) Filed: Dec. 10, 2025

Related U.S. Application Data
(60) Provisional application No. 63/730,016, filed on Dec. 10, 2024.

(51) Int. Cl. A61B 50/33; A61B 50/30

(52) U.S. Cl. CPC A61B 50/33; A61B 2050/3008

(57) ABSTRACT

A surgical tray or surgical case is divided into compartments having foam inserts with letters marking the various compartments. During a surgical procedure the use of the compartments provides an efficient way to identify tools.

This application claims the benefit of U.S. Provisional Patent Application Ser. No. 63/730,016 filed Dec. 10, 2024, the contents of which are incorporated herein by reference.

### BACKGROUND

### 1. Field

This application is directed to surgical trays, in particular, the application is directed to surgical trays that may be formed as a tray or folded as a case that opens into a tray. These trays are used to make surgical procedures more efficient and less prone to error.

### 2. Description of the Related Art

Surgical trays are medical devices used in operating rooms and clinical environments to organize, store, and transport surgical instruments and accessories. These trays play a role in maintaining the sterility and accessibility of surgical tools during medical procedures. Typically constructed from durable materials such as stainless steel, anodized aluminium, or high-grade polymers, surgical trays are designed to withstand repeated sterilization cycles involving autoclaving, chemical disinfection, or other cleaning processes.

Conventional surgical trays are often configured with compartments, slots, or racks that secure instruments in place, preventing movement and potential contamination during transport or handling. While traditional trays provide basic functionality, they lack the ability to manage surgical equipment efficiently during procedures.

Currently, during surgery, items are either left on the trays or if removed, placed on the back table or mayo stand. The problem with this method is that as surgeries are performed, items are misplaced or dropped due to clutter and many different items being moved around on the back table or mayo. Having to locate different surgical items and differentiate between different sizes adds time to procedures which raises costs of surgeries for the hospital.

Therefore, there is a need to provide an effective tray for surgical procedures.

### SUMMARY

Briefly described, a surgical tray has a predetermined area that houses a surgical grade foam that provides easy identification of disposable and semi disposable surgical items.

An aspect of the disclosure is a surgical tray. The surgical tray comprising a first portion having a first top surface and a first bottom surface; a second portion having a second top surface and a second bottom surface; a canal located between the first portion and the second portion; wherein the first top surface is divided into a first plurality of primary compartments, wherein at least one of the first plurality of compartments has a foam insert; wherein the second top surface is divided into a second plurality of primary compartments, wherein at least one of the second plurality of primary compartments has another foam insert; and wherein the foam insert has a character placed thereon and the another foam insert has another character placed thereon different than the character on the foam insert.

Another aspect of the disclosure is a surgical tray system. A surgical tray system comprising a first portion having a first top surface and a first bottom surface; a second portion having a second top surface and a second bottom surface; a canal connecting the first portion and the second portion; wherein the first top surface is divided into a first plurality of primary compartments, wherein at least one of the first plurality of primary compartments has a foam insert; wherein the second top surface is divided into a second plurality of compartments, wherein at least one of the second plurality of primary compartments has another foam insert; wherein the foam insert has a character placed thereon and the another foam insert has another character placed thereon; and a surgical item adapted to be placed within at least one of the plurality of first primary compartments and the plurality of second primary compartments.

Still yet another aspect of the disclosure is a method for implementing a surgical tray. The method comprising placing a surgical item on at least one of a foam insert or another foam insert, wherein the foam insert or the another foam insert are part of a surgical tray comprising; a first portion having a first top surface and a first bottom surface; a second portion having a second top surface and a second bottom surface; a canal extending between the first portion and the second portion; wherein the first top surface is divided into a first plurality of primary compartments, wherein at least one of the first plurality of primary compartments has the foam insert; wherein the second top surface is divided into a second plurality of compartments, wherein at least one of the second plurality of primary compartments has the another foam insert; wherein the foam insert has a character placed thereon and the another foam insert has another character placed thereon.

### BRIEF DESCRIPTION OF THE DRAWINGS

The foregoing summary, as well as the following detailed description of illustrative embodiments of the present application, will be better understood when read in conjunction with the appended drawings. Illustrating the present application are the drawings that depict the embodiments of the disclosure. It should be understood, however, that the application is not limited to the precise arrangements and instrumentalities shown in the drawings.

FIG. 1 is a top-down view of a surgical tray;

FIG. 2 is top-down plan view of the surgical tray;

FIG. 3 is a cross-sectional view of the surgical tray taken along the line A-A;

FIG. 4 is top-down view of the surgical tray case; and

FIG. 5 is a side view of the surgical tray case.

### DETAILED DESCRIPTION

To facilitate an understanding of embodiments, principles, and features of the present disclosure, they are disclosed hereinafter with reference to implementation in illustrative embodiments. Embodiments of the present disclosure, however, are not limited to use in the described treatments or methods and may be utilized in other treatments and methods as will be understood by those skilled in the art familiar with this disclosure.

The components described hereinafter as making up the various embodiments are intended to be illustrative and not restrictive. Many suitable components that would perform the same or a similar function as the components described herein are intended to be embraced within the scope of embodiments of the present disclosure.

The following detailed description refers to the accompanying drawings. Wherever possible, the same reference numbers are used in the drawings and the following description to refer to the same or similar elements. While embodiments may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the claimed subject matter. Instead, the proper scope of the claimed subject matter is defined by the appended claims.

The present disclosure relates to a surgical tray or case configured to facilitate efficient organization and retrieval of surgical items in an operating room environment. The surgical tray or case enable an operating room nurse or other medical personnel to systematically arrange disposable and semi-disposable surgical components required at the commencement of a surgical procedure and throughout its duration. By providing structured organization, the surgical case improves workflow efficiency and enhances safety by permitting rapid access to surgical items prior to and during the procedure. In certain embodiments, the surgical case may also incorporate features for contamination control, ergonomic handling, and modular adaptability to accommodate different surgical specialties.

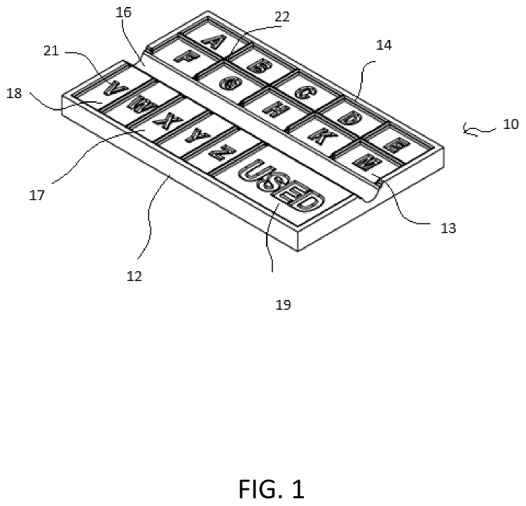

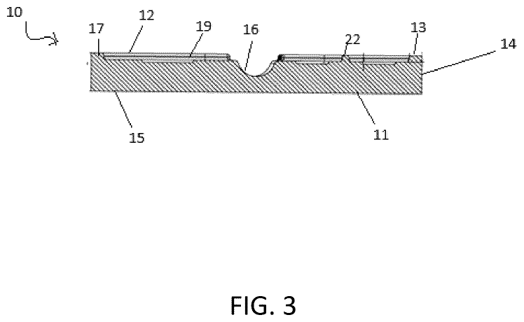

In an embodiment, illustrated in FIGS. 1-3, the surgical tray 10 comprises a rigid body formed of a solid polymeric material, such as medical-grade plastic. Alternative embodiments may employ composite materials, stainless steel, or reinforced polymers to provide enhanced durability and sterilization compatibility.

The surgical tray 10 includes a first portion 12 and a second portion 14 separated by a channel 16. Further discussed below is an alternative embodiment that implements a hinge to enable closing of a surgical case. Such closing mechanisms can be a hinge or magnetic coupling mechanism. In some embodiments, the first portion 12 and second portion 14 may be detachable to allow independent use or sterilization.

The first portion 12 defines a first top surface 17 and a first bottom surface 15, while the second portion 14 defines a second bottom surface 11 and a second top surface 13. The first top surface 17 and the second top surface 13 collectively form a plurality of compartments, including primary compartments 18 and a disposal compartment 19, separated by dividers 22. When reference is made to the first top surface 17 and second top surface 13 and first bottom surface 13 and second bottom surface 11 it should be understood that “top” and “bottom” are referring to the orientation of the portions of the surgical tray 10 where instruments may be placed. Generally, instruments will be placed on the first top surface 17 and second top surface 13.

Still referring to FIGS. 1-3, primary compartments 18 are generally adapted to receive surgical equipment. In an embodiment, additional compartments may be provided for specialized instruments, sutures, or implantable devices. A character region 21 may be provided for labelling or identification purposes. In the embodiment shown, letters are used. In an embodiment, numbers can be used. In an embodiment, icons can be used that are representative of different animals or objects. In an embodiment, the character region 21 can include printed, embossed, or electronic display elements such as RFID tags or QR codes for digital inventory tracking.

A canal 16 is disposed between the first portion 12 and the second portion 14. In various embodiments, the canal 16 may be configured as a beveled channel, a duct, or a trough. The canal 16 extends the length of the first portion 12 and the second portion 14.

In an embodiment, the canal 16 has a “U” shaped cross-section. In an embodiment, the canal 16 has a semi-circle cross-section. In an embodiment, the canal 16 has a semi-rectangular cross-section. In an embodiment, the canal 16 extends the length of the surgical tray 10. In an embodiment, the canal 16 extends less than the length of the surgical tray 10.

The canal 16 can provide storage for tubing and other items that are elongated. The canal 16 may serve as a conduit for fluid drainage, instrument placement, or structural reinforcement. In certain embodiments, the canal 16 may incorporate antimicrobial coatings or detachable liners to maintain sterility. The canal 16 may be dimensioned to accommodate specific surgical tools or accessories, such as suction tubing, electrocautery cords, or laparoscopic instruments, thereby enhancing the functional versatility of the surgical case. In some embodiments, the canal 16 may include integrated heating or cooling elements to maintain temperature-sensitive items.

The first top surface 17 and second top surface 13 each include primary compartments 18 adapted to receive surgical instruments and consumables. These primary compartments 18 may incorporate medical-grade foam inserts. In an embodiment, the primary compartments 18 may be contoured to securely retain items in a predetermined orientation. In an embodiment, the primary compartments 18 may include foam inserts that include raised or recessed characters formed thereon to provide visual or tactile indicators for item identification, thereby reducing retrieval time and minimizing procedural errors.

Types of medical grade foam that can be used are polyurethane foam, silicone foam, polyethylene foam, polyvinyl alcohol foam, latex foam, polycarbonate foam, ethylene vinyl acetate foam, alginates or hydrocolloid foams, polyimide foam, viscoelastic foam, bio-based and biodegradable foam.

In an embodiment, the medical grade foam has character regions 21. The lettering or characters in the character regions 21 facilitate providing organization prior to and during medical procedures. The medical grade foam in an embodiment that is used to hold items used during surgical procedures. For example, some of the medical items placed on the compressed foam may be wires, pins, drills, and reamers. The placement of the items is such that they are able to be utilized quickly and efficiently. The case can aid scrub techs, doctors, vendors, nurses, and charge nurse by facilitating the organization of the items.

Foam is used to permit a user to easily mark the foam with a marker or other writing implement to identify the items that are going to be placed within each compartment.

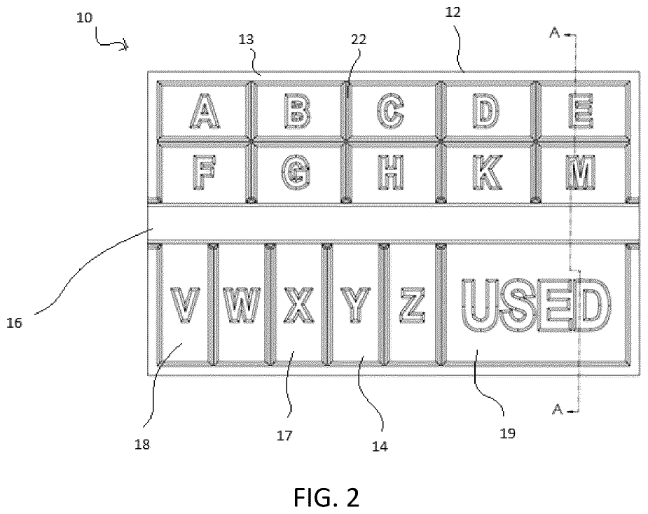

In FIGS. 1 and 2, the letters A, B, C, D, and E are placed in one row of the surgical tray 10. In the second row of the surgical tray 10 are the letters F, G, H, K, and M. On the opposite side of the surgical tray 10 are the letters V, W, X, Y, and Z. Also in that row is the word USED. Some of the letters are in alphabetical order, while other letters are not. Selection of letters is made based upon the ease of identifying letters during surgical procedures, both visually and auditorily, or at least one of the two. Thes election of the letters used to mark the foam insert are chosen because of the ease of identification and the ability to distinguish the spoken letter from other letters during a surgical procedure.

In an embodiment, characters of the Latin alphabet are used. In an embodiment, characters from the Cyrillic alphabet are used. In an embodiment, characters from an alphabet selected from one or more of the following alphabets are used Latin, Phoenician, Cyrillic, Greek, Arabic, and Hebrew. In an embodiment, symbols from Chinese, Japanese, Korean, or Thai alphabets are used. In an embodiment, numbers are used. In an embodiment, abstract characters or pictograms are used. In an embodiment, images of animals, objects, people, or vegetation are used.

In the embodiment shown in FIGS. 1 and 2, fifteen primary compartments 18 and a disposal compartment 19 are shown. In an embodiment, more than fifteen primary compartments 18 can be used. In an embodiment, less than fifteen primary compartments 18 can be used. In an embodiment, more than one disposal compartment 19 can be used. In an embodiment, less than one disposal compartment 19 can be used.

In an embodiment, the primary compartments 18 are all equally sized. In an embodiment, some primary compartments 18 are sized the same as others, while other primary compartments 18 are of different size. In the embodiment shown in FIGS. 1 and 2, ten of the primary compartments 18 are the same size, five primary compartments 18 are smaller than the ten primary compartments 18 and one disposal compartment 19 is larger than all the others.

Alternative embodiments may employ adjustable dividers, spring-loaded clips, or magnetic retention systems to accommodate instruments of varying sizes. In some configurations, the compartments may include transparent covers or sterile barriers to prevent contamination while maintaining visibility. Additionally, electronic sensors may be integrated within the compartments to detect the presence or absence of critical instruments, providing real-time feedback to operating room personnel.

The surgical tray 10 set forth in this disclosure allows a nurse in the operating room to efficiently organize both disposable and semi-disposable surgical items that may be needed during a surgical procedure and at the start of the procedure. The efficient organization permits the nurse to readily and safely access the surgical items that will be used prior to and during the procedure.

To implement use of the surgical tray 10 during a procedure, the operating room staff begins by bringing into the surgery room the surgical tray 10. Each primary compartment 18 is lined with medical-grade foam inserts that are pre-marked in the character regions 21 with letters for quick identification. Prior to the start of surgery, disposable and semi-disposable surgical items—such as wires, pins, drills, and reamers—are placed into designated primary compartments 18 according to the labeling scheme. This structured arrangement ensures that every item is organized and easily accessible.

During the procedure, the scrub technician or nurse retrieves instruments directly from the surgical tray 10 without searching through cluttered surfaces. The foam inserts hold items securely in a predetermined orientation, reducing the risk of contamination or accidental dropping. If an item is used and needs to be set aside, it can be placed in a dedicated disposal compartment clearly marked for that purpose. This minimizes confusion and prevents mixing used instruments with sterile ones.

After the procedure, the surgical tray 10 can be quickly audited by checking the primary compartments 18 to confirm that all instruments have been accounted for. The foam inserts allow for easy marking or customization, enabling staff to adapt the tray for different surgical specialties or case types. This method improves workflow efficiency, reduces procedural delays, and enhances patient safety by maintaining a clear, organized environment throughout the surgery.

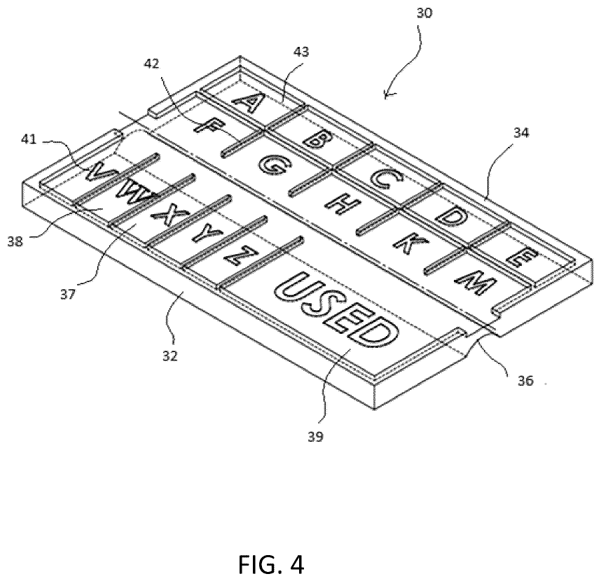

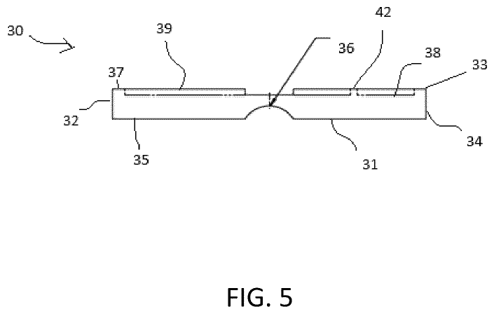

Turning to FIGS. 4 and 5, shown is a surgical case 30 that functions in a similar manner as the surgical tray 10. The surgical case 30 comprises a rigid body formed of a solid polymeric material, such as medical-grade plastic. Alternative embodiments may employ composite materials, stainless steel, or reinforced polymers to provide enhanced durability and sterilization compatibility.

The surgical case 30 includes a first portion 32 and a second portion 34 separated by a hinge 36. Alternatives to the hinge 36 may be used, such as closing mechanisms like a magnetic coupling mechanism. In some embodiments, the first portion 32 and second portion 34 may be detachable to allow independent use or sterilization.

The first portion 32 defines a first interior surface 37 and a first exterior surface 35, while the second portion 34 defines a second exterior surface 31 and a second interior surface 33. The first interior surface 37 and the second interior surface 33 collectively form a plurality of compartments, including primary compartments 38 and a disposal compartment 39, separated by dividers 42.

When reference is made to the first interior surface 37 and second interior surface 33 and first exterior surface 33 and second exterior surface 31 it should be understood that is referring to the orientation of the portions of the surgical case 30 where instruments may be placed. Generally, instruments will be placed on the first interior surface 37 and second interior surface 33.

A hinge 16 is located between the first portion 32 and the second portion 34. Located in the first interior portion 37 and the second interior portion 33 are primary compartments 38. The primary compartments 38 contain medical grade foam that has formed thereon character regions 41.

Referring now to the figures, in an embodiment, and as shown in FIGS. 4 and 5, the hinge 36 is a living hinge. In an embodiment, the hinge is a piano hinge. In an embodiment, the hinge is a butt hinge. In an embodiment, the hinge is a concealed hinge. In an embodiment, the hinge is a barrel hinge. In an embodiment, the hinge is a box hinge. In an embodiment, the hinge is a T-Hinge. In an embodiment, the hinge is a spring hinge. In an embodiment, the hinge is a soft-close hinge. In an embodiment, the hinge is a strap hinge. In an embodiment, the hinge is a pivot hinge.

FIG. 4 is top-down view of the surgical case 30 opened to form a tray. The surgical case 30 has a medical grade foam inserted into the compartments of the case when it is opened and in tray form. Lettering or characters are placed on the foam.

In the embodiment shown in FIG. 4, fifteen primary compartments 38 are shown. In an embodiment, more than fifteen primary compartments can be used. In an embodiment, less than fifteen primary compartments can be used.

In an embodiment, the primary compartments 38 are all equally sized. In an embodiment, some primary compartments 38 are sized the same as others, while other primary 38 compartments are of different size. In the embodiment shown in FIG. 4, ten of the primary compartments 38 are the same size, five primary compartments 38 are smaller than the ten compartments and one disposal compartment 39 is larger than all the others.

In FIG. 4, the letters A, B, C, D, and E are placed in one row of the surgical case in the second portion 34. In the second row of the second portion 34 are the letters F, G, H, K, and M. On the other side of the surgical case 30 in the first portion 32 are the letters V, W, X, Y, and Z. Further in that row is the word USED in the disposal compartment 39. Some of the letters are in alphabetical order, while other letters are not.

In FIG. 4, the selection of the letters used to mark the foam insert are chosen because of the ease of identification and the ability to distinguish the spoken letter from other letters during a surgical procedure.

In an embodiment, characters of the Latin alphabet are used. In an embodiment, characters from the Cyrillic alphabet are used. In an embodiment, characters from an alphabet selected from one or more of the following alphabets are used Latin, Phoenician, Cyrillic, Greek, Arabic, and Hebrew. In an embodiment, symbols from Chinese, Japanese, Korean, or Thai alphabets are used. In an embodiment, numbers are used. In an embodiment, abstract characters or pictograms are used. In an embodiment, images of animals, objects, people, or vegetation are used.

FIG. 5 is a side view of the surgical tray 30 showing the living hinge 36.

Wherever possible, the same or like reference numbers are used throughout the drawings to refer to the same or like features. It should be noted that the drawings are in simplified schematic form and are not drawn to precise scale. Certain terminology used in the description is for convenience only and is not limiting. The term “a,” as used in the specification, means “at least one.” The terminology includes the words above specifically mentioned, derivatives thereof, and words of similar import.

Furthermore, the described features, advantages and characteristics of exemplary embodiments may be combined in any suitable manner in one or more embodiments. One skilled in the art will recognize, in light of the description herein, that the exemplary embodiments can be practiced without one or more of the specific features or advantages of a particular embodiment. In other instances, additional features and advantages may be recognized in certain embodiments that may not be present in all embodiments of the present disclosure.

While embodiments of the present disclosure have been disclosed in exemplary forms, it will be apparent to those skilled in the art that many modifications, additions, and deletions can be made therein without departing from the spirit and scope of the invention and its equivalents, as set forth in the following claims.

What is claimed is:

1. A surgical tray comprising:
a first portion having a first top surface and a first bottom surface;
a second portion having a second top surface and a second bottom surface;
a canal located between the first portion and the second portion;
wherein the first top surface is divided into a first plurality of primary compartments, wherein at least one of the first plurality of compartments has a foam insert;
wherein the second top surface is divided into a second plurality of primary compartments, wherein at least one of the second plurality of primary compartments has another foam insert; and
wherein the foam insert has a character placed thereon and the another foam insert has another character placed thereon different than the character on the foam insert.

2. The surgical tray of claim 1, wherein the canal extends the length of the first portion.

3. The surgical tray of claim 1, wherein the canal has a semi-circle cross-section.

4. The surgical tray of claim 1, wherein the first plurality of primary compartments are equally sized.

5. The surgical tray of claim 1, wherein the second plurality of primary compartments are not equally sized.

6. The surgical tray of claim 1 wherein each of the second plurality of primary compartments are differently sized than the first plurality of primary compartments.

7. The surgical tray of claim 1, wherein the character on the foam insert is from the Latin alphabet.

8. The surgical tray of claim 1, further comprising a plurality of additional characters placed on the foam insert and another foam insert, wherein at least some of the plurality of additional characters are not in alphabetical order.

9. A surgical tray system comprising:
a surgical tray comprising;
a first portion having a first top surface and a first bottom surface;
a second portion having a second top surface and a second bottom surface;
a canal connecting the first portion and the second portion;
wherein the first top surface is divided into a first plurality of primary compartments, wherein at least one of the first plurality of primary compartments has a foam insert;
wherein the second top surface is divided into a second plurality of compartments, wherein at least one of the second plurality of primary compartments has another foam insert;
wherein the foam insert has a character placed thereon and the another foam insert has another character placed thereon; and
a surgical item adapted to be placed within at least one of the plurality of first primary compartments and the plurality of second primary compartments.

10. The surgical tray system of claim 9, wherein the canal extends the length of the first portion.

11. The surgical tray system of claim 9, wherein the canal has a semi-circle cross-section.

12. The surgical tray system of claim 9, wherein the first plurality of primary compartments are equally sized.

13. The surgical tray system of claim 9, wherein the second plurality of primary compartments are not equally sized.

14. The surgical tray system of claim 9, wherein each of the second plurality of primary compartments are differently sized than the first plurality of primary compartments.

15. The surgical tray system of claim 9, wherein the character on the foam insert is from the Latin alphabet.

16. The surgical tray of claim 9, further comprising a plurality of additional characters placed on the foam insert and another foam insert, wherein at least some of the plurality of additional characters are not in alphabetical order.

17. A method for implementing a surgical tray comprising:
placing a surgical item on at least one of a foam insert or another foam insert, wherein the foam insert or the another foam insert are part of a surgical tray comprising;
a first portion having a first top surface and a first bottom surface;
a second portion having a second top surface and a second bottom surface;
a canal extending between the first portion and the second portion;
wherein the first top surface is divided into a first plurality of primary compartments, wherein at least one of the first plurality of primary compartments has the foam insert;
wherein the second top surface is divided into a second plurality of compartments, wherein at least one of the second plurality of primary compartments has the another foam insert; and
wherein the foam insert has a character placed thereon and the another foam insert has another character placed thereon.

18. The method of claim 17, wherein the canal extends the length of the first portion.

19. The method of claim 17, wherein the canal has a semi-circle cross-section.

20. The method of claim 17, wherein the first plurality of primary compartments are equally sized.
